(19) United States
(12) Patent Application Publication
(10) Pub. No.: US 2026/0151065 A1
(43) Pub. Date: Jun. 4, 2026

(54) METHOD OF DEPRESSION DETECTION AND SYSTEM THEREOF

(71) Applicant: National Cheng Kung University, Tainan (TW)

(72) Inventors: Mei-Feng LIN, Tainan (TW); Hong-Ju SHEN, Tainan (TW); Chia-Feng HSU, Tainan (TW); Yi-Chien PAN, Tainan (TW); Fei-Pi LIU, Tainan (TW)

(21) Appl. No.: 19/404,243

(22) Filed: Dec. 1, 2025

Related U.S. Application Data
(60) Provisional application No. 63/727,674, filed on Dec. 4, 2024.

(51) Int. Cl. A61B 5/16; G06N 20/00; G06V 40/16

(52) U.S. Cl. CPC A61B 5/165; G06N 20/00; G06V 40/176

(57) ABSTRACT

The present disclosure provides a method for depression detection and a system thereof, wherein the method comprises the following steps: a face marking step, an emotion classification and calculation step, a depression identification step, and a verification step. The method for depression detection also comprises a semantic analysis step and an integrated depression step to determine a level of depression. The method for depression detection has high accuracy and stability in predicting facial emotions of women with breast cancer, and its results show a significant correlation with traditional depression scales and are more sensitive to subtle emotions.

### CROSS-REFERENCE TO RELATED APPLICATION

The present application is related to and claims the benefit of U.S. Provisional Application No. 63/727,674, filed Dec. 4, 2024. The aforementioned application is hereby incorporated by reference in its entirety.

### FIELD OF THE INVENTION

The present invention relates to a system and method for detection technologies, specifically to a system and method for detecting depression.

### BACKGROUND OF THE INVENTION

In all kinds of cancers, women with breast cancer have the longest survival rate and will confront physical, emotional, social, and financial problems after chemotherapy, and because of their cognitive decline and higher psychological distress comprising: anxiety, depression, stress, and worry, a long-term emotional monitoring is necessary in order to improve the quality of women with breast cancer's life after surgery.

Assessment methods of the prevalence of depression include clinical diagnosis, use of antidepressants, depression scales, and norm-referenced tests, wherein tools with self-assessment show a higher prevalence of depression, while clinical diagnosis methods show a lower one. The results indicated that using a single questionnaire assessment method may overestimate depression and lead to biases. By contrast, depression scales, including the Hamilton Depression Rating Scale and the Beck Depression Inventory, are commonly used and more suitable for screening, but have no clinical diagnostic functions.

Emotion recognition can be performed based on various features, such as facial expressions and language, where facial expressions are clearly visible and exhibit multiple distinct features. In addition, facial expressions exhibit significant consistency across different ethnic and cultural backgrounds, resulting in a vast database that is widely used.

The Facial Action Coding System, based on Anatomy, analyzes subtle expressions and emotional features according to Action Units, which are divided by facial muscle movements. Currently, by applying technology and biometric indicators, six common facial emotions—happy, sad, anger, surprise, fear, and disgust—can be detected automatically, and an artificial intelligence-based emotional analysis technique is subsequently developed.

Because languages from various cultural backgrounds differ, semantic and emotional analysis needs to consider the vocabulary size in the semantic dictionaries compiled by research institutions in each country. For instance, the most complete Traditional Chinese semantic emotional dictionary contains 26,021 words, but it still falls short in detecting the emotional features of depression. Therefore, a more comprehensive semantic database specifically for depression is required to enhance the accuracy and clinical practicality of emotion recognition.

However, the assessment method of depression currently relies on questionnaires and psychological interviews, and an emotion recognition system and prediction model based on physiological index remain absent in the optimization of emotional assessment in breast cancer patients. Furthermore, the accuracy of emotion recognition systems is currently inconsistent, and the efficiency of detecting emotions is uncertain. The depression model in the prior art is primarily established using microdata from video databases, but it is only aimed at a single feature, comprising semantics, volume, or a single image, during depression recognition.

### SUMMARY OF THE INVENTION

The present invention achieves technical advantages as a system and method for depression detection in which a subject's possible presence or symptoms of facial depression can be predicted.

In some embodiments, a depression detection system comprises: an emotional classification model configured to read a plurality of facial features to generate a plurality of corresponding depression coordinates, and calculate the plurality of corresponding depression coordinates in accordance with a standard coordinate to generate a plurality of features; and an analysis model configured to read a highest correlated feature to determine a facial depression level.

In some embodiments, the plurality of facial features is obtained by marking a subject's facial image sequence, which is a video recording processed frame by frame.

In some embodiments, the highest correlated feature is a statistical feature that exhibits the highest correlation with a depression measurement tool, and this statistical feature is derived through a statistical analysis of the plurality of features.

In some embodiments, the facial depression level comprises depressed and non-depressed, and while the facial depression level is the non-depressed, a suppression measurement tool is provided to determine an emotional suppression level of the subject, wherein the facial depression level is true when the emotional suppression level shows no high emotional suppression.

In some embodiments, the depression detection system further comprises: a semantic analysis model configured to read a semantic record and generate a semantic depression level; and an integrated model configured to read the facial depression level and the semantic depression level to generate an integrated depression level.

In some embodiments, the semantic record is obtained by processing the video recording.

In some embodiments, when the facial depression level is the non-depressed and the emotional suppression level shows high emotional suppression, a weight of the semantic depression level is larger than a weight of the facial depression level.

In some embodiments, the video recording is a frontal video of the subject describing self-mentation for a duration, and the duration is 5 to 30 minutes.

In some embodiments, the emotional classification model comprises a Valence-Arousal model, a Valence-Arousal model with POSTER++, a Multimodal model, a Convolutional Neural Network, a Long Short-Term Memory model, or any combination of two or more thereof.

In some embodiments, the standard coordinate comprises: a first coordinate, a second coordinate, a third coordinate and a fourth coordinate, wherein the first coordinate combines and relocates a coordinate of basic emotion Happy with a coordinate of basic emotion Surprise; the second coordinate, the third coordinate and the fourth coordinate correspond to a coordinate of basic emotion Neutral, a coordinate of basic emotion Sad and a coordinate of basic emotion Anger, respectively.

In some embodiments, the depression measurement tool comprises a Hamilton Depression Rating Scale, a Beck Depression Inventory, a Patient Health Questionnaire, or a Taiwanese Depression Scale.

In some embodiments, the suppression measurement tool comprises: a Courtauld Emotional Control Scale, an Emotional Regulation Questionnaire, or an Emotional Expressivity Scale.

In some embodiments, the analysis model comprises an Ensemble Voting Classifier, a Random Forest model, a Multilayer Perceptron, a Decision Tree, a Support Vector Machine, an Artificial Neural Network, a Convolutional Neural Network, or any combination of two or more thereof.

In some embodiments, the semantic analysis model comprises an Event-Driven Depression Tendency Warning model, an Event-Driven Depression Tendency Warning model version II or a Python senti_c package.

The integrated model comprises a Gaussian Process Regression model or a Bayesian Neural Network.

In some embodiments, the method of obtaining the plurality of facial features comprises using a Convolutional Neural Network, an Open Computer Vision Library, a Py-Feat, an OpenFace, an Active Appearance Model, or any combination of two or more thereof.

The depression detection method and system thereof provided in the present invention exhibits a high accuracy and stability in facial emotional prediction of breast cancer patients, and the result of the depression detection, with a significant relevance to traditional assessment of depression, shows higher sensitivity in subtle emotions.

### BRIEF DESCRIPTION OF THE DRAWINGS

FIG. 1 is a flowchart illustrating an embodiment of the depression detection process.

FIG. 2 is a flowchart of another embodiment illustrating the process of depression detection.

FIG. 3 is a coordinate map illustrating the valence-arousal coordinate.

FIG. 4 is a flowchart of the depression detection system.

FIG. 5 is a diagram illustrating the method and content of obtaining the video recording.

FIG. 6 illustrates the motion classification and calculation step.

### DETAILED DESCRIPTION OF THE INVENTION

Hereinafter, embodiments will be described with reference to the drawings. However, the embodiments can be implemented with many different modes, and it will be readily appreciated by those skilled in the art that modes and details thereof can be changed in various ways without departing from the spirit and scope thereof. Thus, the present invention should not be interpreted as being limited to the following description of the embodiments.

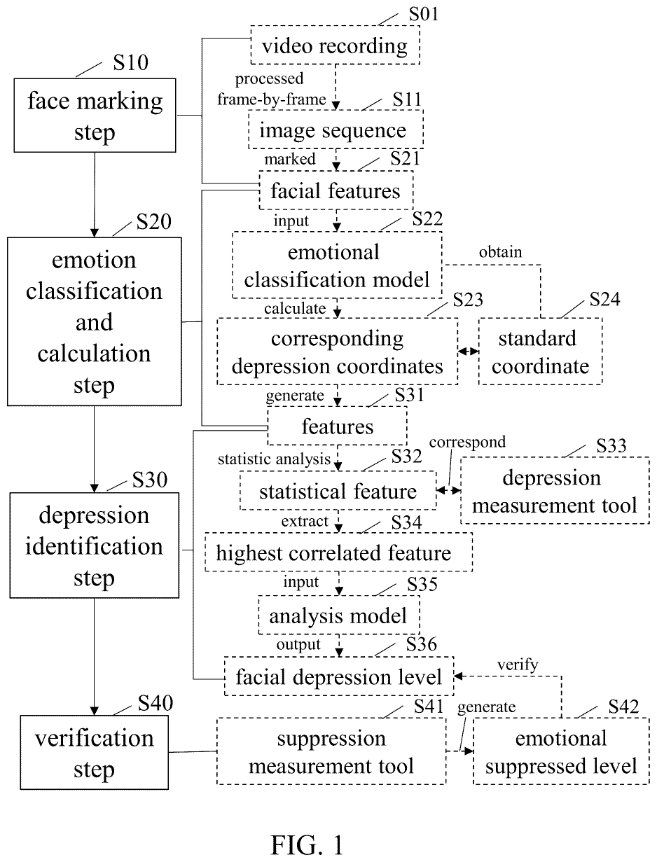

Referring to FIG. 1, an embodiment of the method for depression detection comprises steps of:
a face marking step S10: marking a facial image to obtain a plurality of facial features S21, wherein the facial image is an image sequence S11 obtained by processing a subject's video recording S01 frame by frame;
an emotion classification and calculation step S20: inputting the plurality of facial features S21 into an emotional classification model S22 to generate a plurality of corresponding depression coordinates S23, and calculating the plurality of corresponding depression coordinates S23 in accordance with a standard coordinate S24 to generate a plurality of features S31;
a depression identification step S30: performing a statistical analysis on the plurality of features S31 to generate a statistical feature S32, extracting the statistical feature S32 having the highest correlation with a depression measurement tool S33 as a highest correlated feature S34, and inputting the highest correlated feature S34 into an analysis model S35 to generate a facial depression level S36 comprising: depressed and non-depressed; and
a verification step S40: when the facial depression level S36 is the non-depressed, determining an emotional suppression level S42 of the subject by a suppression measurement tool S41, wherein the facial depression level S36 is true when the emotional suppression level S42 shows no high emotional suppression; however when the emotional suppression level S42 shows high emotional suppression, the non-depressed of the facial depression level S36 may be underestimated and needs further determination.

In various embodiments, the pattern of manifestation of the depression further comprises a degree, number, percentage, score, or chart, but is not limited to those above. Any methods or tools that can differentiate the subject's depression level are feasible.

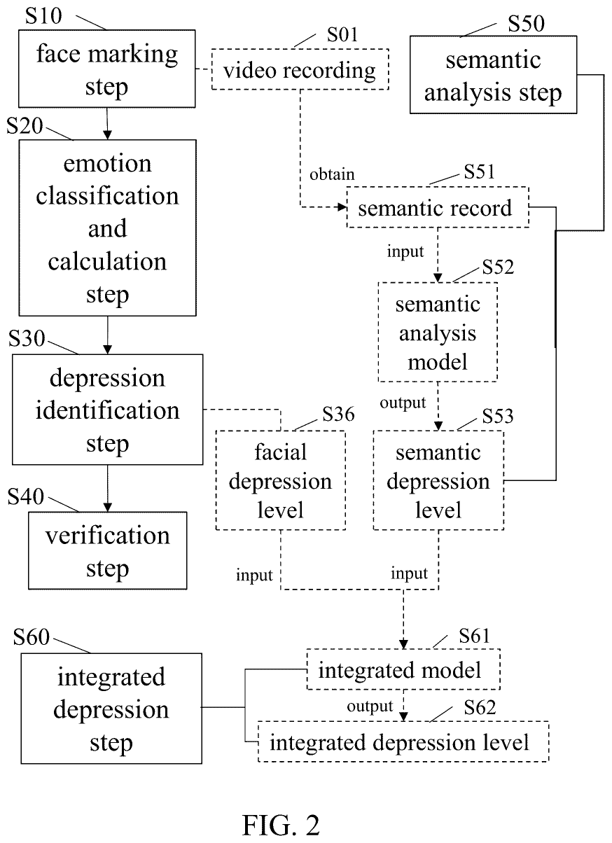

Referring to FIG. 2, in one embodiment, the video recording S01 further comprises a semantic record S51, and the method for depression detection further comprises steps of:
a semantic analysis step S50: inputting the semantic record S51 into a semantic analysis model S52 to generate a semantic depression level S53; and
an integrated depression step S60: inputting the facial depression level S36 and the semantic depression level S53 into an integrated model S61 to generate an integrated depression level S62, wherein when the facial depression level S36 is the non-depressed and the emotional suppression level S42 shows high emotional suppression, a weight of the semantic depression level S53 is larger than weight of the facial depression level S36.

In various embodiments, the video recording S01 is a frontal video of the subject describing self-mentation for a duration, and the duration may be 5 to 10 minutes, 10 to 15 minutes, 15 to 20 minutes, 20 to 25 minutes, or 25 to 30 minutes, and 10 minutes is a preferred option for the length of the duration.

In addition, a sampling period of processing the video recording S01 frame by frame may be 0.5 to 1 second, 1 to 1.5 seconds, 1.5 to 2 seconds, 2 to 2.5 seconds, 2.5 to 3 seconds, 3.5 to 4 seconds, 4 to 4.5 seconds, or 4.5 to 5 seconds, and 1 second is a preferred option for the sampling period.

The descriptive contents of the subject's self-mentation may include: a narrative of stressful events in a preceding period, wherein a time duration of the preceding period is not limited, and a preferred option for the time duration may be in a range of one to two weeks or determined according to evaluation by professionals in the art.

Additionally, the subject can classify the stressful events and visualize or quantify their emotional state with the assistance of an application on a smartphone, a Brief Symptom Rating Scale, or among other tools.

In some embodiments, the facial image comprises a position of the subject's face, landmarks of the subject's face, or a combination thereof, with the latter being a preferred option for the facial image.

In addition, the method of obtaining the plurality of facial features S21 comprises: a Convolutional Neural Network, an Open Computer Vision Library, a Py-Feat, an OpenFace, an Active Appearance Model or any combination of two or more thereof but not limited to those tools above, any machine learning models or tools that can mark the facial features is feasible; the Convolutional Neural Network is a preferred option for the method of obtaining the plurality of facial features S21.

As used herein, the term “image sequence” means arrangement of the image is in a regular time series, and because the image is a sequence, any parameters resulted from the image should also be a sequence; for instance, the plurality of facial features S21, the plurality of corresponding depression coordinates S23 and the plurality of features S31 are all in a sequence format.

In various embodiments, the emotional classification model S22 may be a Valence-Arousal model, a Valence-Arousal model with POSTER++, a Multimodal model, a Convolutional Neural Network, a Long Short-Term Memory model or any combination of two or more thereof, but not limited to those tools above, any models or tools that can accurately describe and measure emotion by coordinate system is feasible; the Valence-Arousal model with POSTER++ is a preferred option for the emotional classification model S22.

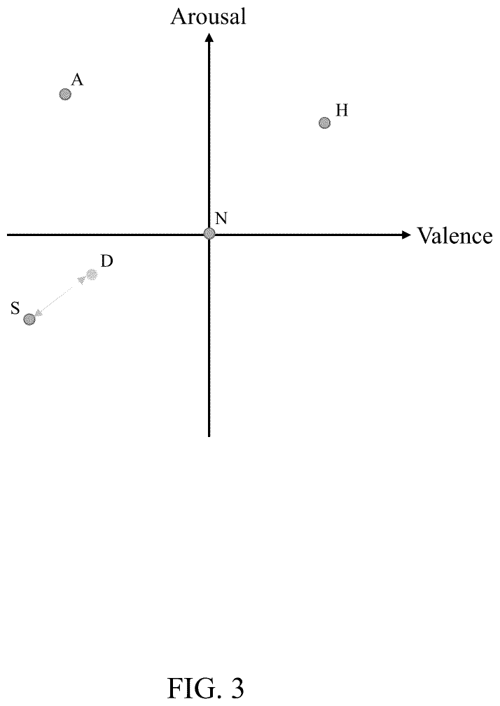

Referring to FIG. 3, the standard coordinate S24 combines seven original basic emotions into four emotions, and the standard coordinate S24 comprises: a first coordinate H, a second coordinate N, a third coordinate S and a fourth coordinate A, wherein the first coordinate H combines and relocates a coordinate of basic emotion Happy with a coordinate of basic emotion Surprise; the second coordinate N, the third coordinate S and the fourth coordinate A correspond to a coordinate of basic emotion Neutral, a coordinate of basic emotion Sad and a coordinate of basic emotion Anger, respectively.

As used herein, the term “coordinate” means valence-arousal coordinate. Referring to FIG. 3, point D represents one of the pluralities of corresponding depression coordinates S23.

Additionally, the plurality of features S31 comprises: a valence coordinate value of point D, an arousal coordinate value of point D, a distance between point D and the standard coordinate S24, and a depression intensity of point D.

In addition, the statistical feature S32 comprises a maximum, a minimum, a mean, a standard deviation, a mode, and a median, but is not limited to these values alone.

In various embodiments, the depression measurement tool S33 may be a Hamilton Depression Rating Scale, a Beck Depression Inventory, a Patient Health Questionnaire, or a Taiwanese Depression Scale, with the Hamilton Depression Rating Scale being the preferred option for the depression measurement tool S33.

In various embodiments, the suppression measurement tool S41 may be a Courtauld Emotional Control Scale, an Emotional Regulation Questionnaire, or an Emotional Expressivity Scale, wherein the Courtauld Emotional Control Scale is a preferred option for the suppression measurement tool S41.

In various embodiment, the analysis model S35 may be an Ensemble Voting Classifier, a Random Forest model, a Multilayer Perceptron, a Decision Tree, a Support Vector Machine, an Artificial Neural Network, a Convolutional Neural Network or any combination of two or more thereof, but not limited to those tools above, any models or tools able to perform classification or regression analysis is feasible; the Random Forest model is a preferred option for the analysis model S35.

In addition, the semantic record S51 may comprise voice data, text data, or a combination thereof, and transferring text data from the voice data is a preferred option for the semantic record S51.

In various embodiments, the semantic analysis model S52 comprises an Event-Driven Depression Tendency Warning model, an Event-Driven Depression Tendency Warning model version II, or a Python senti_c package, wherein the Event-Driven Depression Tendency Warning model version II is a preferred option for the semantic analysis model S52.

As mentioned above, the Event-Driven Depression Tendency Warning model version II incorporates a psychological factor into the development of a five-factor semantic analysis model, alongside other factors, including event, mood, symptom, and thought, thereby improving its accuracy compared to the Event-Driven Depression Tendency Warning model.

In various embodiments, the integrated model S61 may be a Gaussian Process Regression model or a Bayesian Neural Network, but is not limited to these tools. Any models or tools capable of performing classification or regression analysis are feasible; however, the Gaussian Process Regression is a preferred option for the integrated model S61.

In addition, the facial depression level S36, the semantic depression level S53, and the integrated depression level S62 are designated as depressed or non-depressed, respectively.

The emotion classification and calculation step S20 optionally comprises: analyzing a movement of head, eyesight or other emotional classification features in order to further perform weighing and prediction; evaluation of the semantic depression level S53 is based on the semantic data S51 of the image recording S01, wherein the semantic analysis model S52 optionally comprises analyzing tone in order to perform further weighing and prediction.

The depression measurement tool S33 and the suppression measurement tool S41 are assessed and diagnosed by a psychiatrist; besides, the analysis model S35, the semantic analysis model S52, and the integrated model S61, alone or in combination, are compared to an assessment performed by the psychiatrist in order to ensure clinical applicability thereof.

A combination of the facial depression level S36 and the semantic depression level S53 comprises: both indicating the depressed, both indicating the non-depressed, the facial indicating the depressed but the semantic indicating the non-depressed, and the semantic indicating the depressed but the facial indicating the non-depressed; wherein for the subject without high emotional suppression, the subject is determined to be the depressed when the combination is the both indicating the depressed.

In some embodiments, the facial depression level S36 can be classified using a threshold, and preferably, the threshold is a valence coordinate 0.2 and a valence coordinate −0.2; that is to say, when the valence coordinate value of the corresponding depression coordinate S23 is greater than or equal to 0.2, the facial depression level S36 indicates a positive emotion; when the valence coordinate value of the corresponding depression coordinate S23 is less than or equal to −0.2, the facial depression level S36 indicates a negative emotion; and when the valence coordinate value of the corresponding depression coordinate S23 is between −0.2 to 0.2, the facial depression level S36 indicates a neutral emotion.

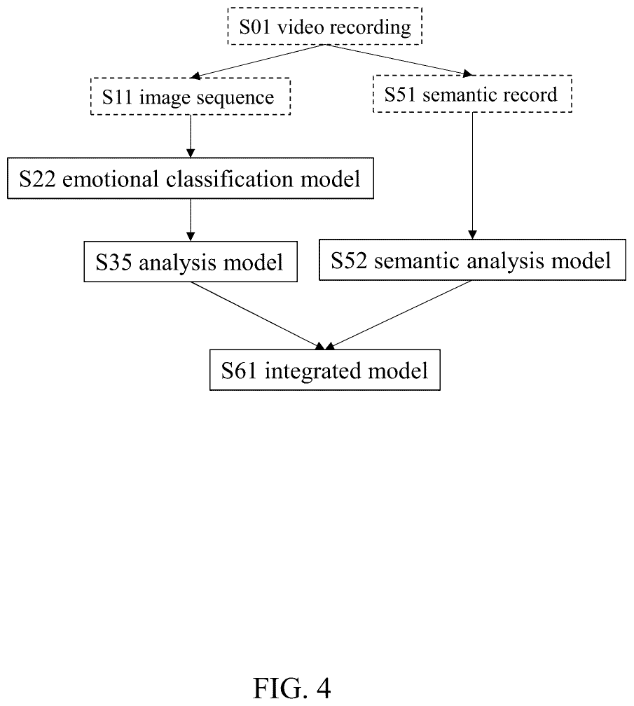

Referring to FIG. 4, an embodiment of the depression detection system comprises:
an emotional classification model S22 configured to read a plurality of facial features to generate a plurality of corresponding depression coordinates, and calculate the plurality of corresponding depression coordinates in accordance with a standard coordinate to generate a plurality of features, wherein the plurality of facial features is obtained by marking an image sequence S11 of a subject's face, which is a video recording S01 processed frame by frame; and
an analysis model S35 configured to read a highest correlated feature to determine a facial depression level, wherein the highest correlated feature is a statistical feature that exhibits the highest correlation with a depression measurement tool, and the statistical feature is derived through a statistical analysis of the plurality of features, wherein the facial depression level comprises: depressed and non-depressed, and while the facial depression level is the non-depressed, a suppression measurement tool is provided to determine an emotional suppression level of the subject, wherein the facial depression level is true when the emotional suppression level shows no high emotional suppression.

Referring to FIG. 4 again, in some embodiments, the depression detection system further comprises:
a semantic analysis model S52 configured to read a semantic record S51 and generate a semantic depression level, wherein the semantic record S51 is obtained from processing the video recording S01; and
an integrated model S61 configured to read the facial depression level and the semantic depression level to generate an integrated depression level, wherein when the facial depression level indicates the non-depressed and the emotional suppression level shows high emotional suppression, a weight of the semantic depression level is larger than a weight of the facial depression level.

### Embodiment 1

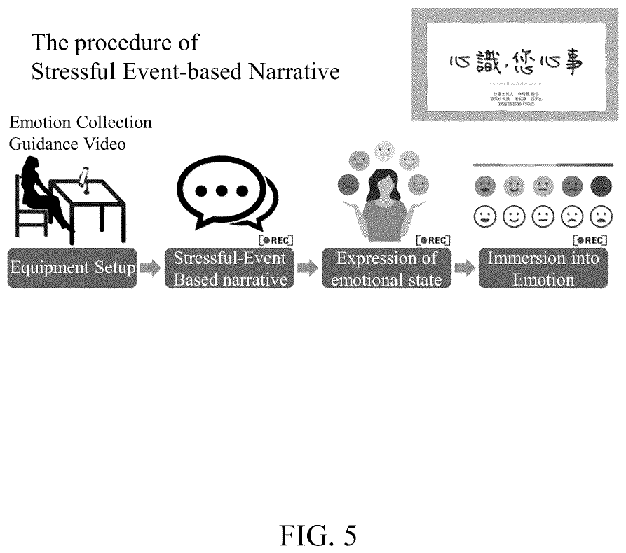

Firstly, referring to FIG. 5, recording equipment is placed at a distance of 50 cm in front of a cancer patient or a mentally ill patient in an isolated space for the purpose of collecting a 10-minute-long frontal video recording of the subject.

The video recording is the subject's narrative of stressful events and self-mentation over the past two weeks, in conjunction with a mobile application and the Brief Symptom Rating Scale.

Additionally, the video recording includes a facial record and a semantic record.

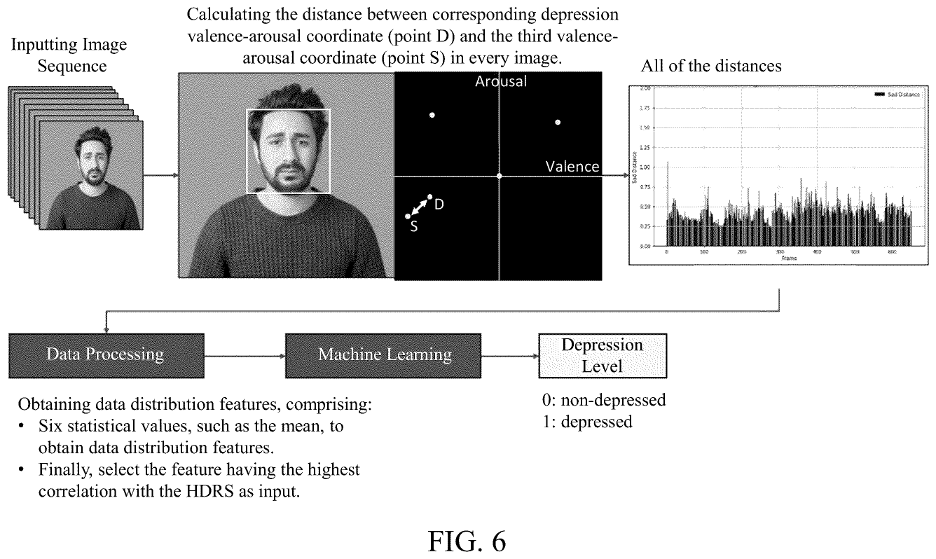

After that, referring to FIG. 6, the facial record is processed into an image sequence of 600 images in a time series, and then input the image sequence into a Convolutional Neural Network to mark a position and landmarks of the patient's face; following, input the position and landmarks of the patient's face into a Valence-Arousal model modified based on POSTER++ structure in order to assess emotion classification, predict valence-arousal coordinate and calculate distance between the valence-arousal coordinate and the valence-arousal coordinate of Sad and depression intensity of the landmarks of the 600 images continuously.

Furthermore, a statistical and regression analysis is performed on the valence-arousal coordinate and the depression intensity to obtain six statistical features, including a maximum, minimum, mean, standard deviation, mode, and median. Correlation of the six statistical features with Hamilton Depression Rating Scale evaluated by the psychiatrist are further assessed to obtain the statistical feature exhibiting the highest correlation with the Hamilton Depression Rating Scale; subsequently, the statistical feature exhibiting the highest correlation with the Hamilton Depression Rating Scale is input into a Random Forest model and Multilayer perceptron to classify facial depression, thereby generating the patient's facial depression level including: depressed or non-depressed.

After that, while the patient is determined to be non-depressed, a Courtauld Emotional Control Scale is required to assess the patient's emotional suppression level. If the subject demonstrates high emotional suppression, the subject's facial depression level may be underestimated and needs to perform a semantic depression analysis and an integrated depression analysis, wherein the weight of the semantic depression level is larger than weight of the facial depression level in the integrated depression analysis; on the other hand, if the facial depression level indicates the depressed, then the verification step, the semantic analysis step and the integrated depression step can be performed alternatively.

Furthermore, the subject's ten-minute semantic record is exported as text data, which is then input into an Event-Driven Depression Tendency Warning model version II to classify the subject's semantic depression level and generate an indication of whether the subject is depressed or non-depressed.

Finally, the facial depression level and the semantic depression level are input into a Gaussian Process Regression model to determine the subject's integrated depression level, including depressed or non-depressed.

To clearly explain the present invention and facilitate better understanding, the method in Embodiment 1 is incorporated into the following system and illustrated in detail.

### Embodiment 2

First, a depression detection system comprises a valence-arousal model with a modified structure based on POSTER++, and the valence-arousal model is able to read position and landmarks of a subject's face of an image sequence encompassing 600 images processed from the subject's 10-minute video record of personal stressful event, and the valence-arousal model also combines seven original basic emotions into four emotions and generate 600 emotional classifications, valence-arousal coordinates, distances between the valence-arousal coordinate and a valence-arousal coordinate of Sad, and depression intensities of the image sequence.

On the other hand, the depression detection system further comprises: a data analysis model and a Random Forest model with Multilayer perceptron, wherein the data analysis model performs statistical and regression analysis on the four generators mentioned above and generates the statistical features as described in embodiment 1; in the following, the Random Forest model with Multilayer perceptron reads the statistical feature having the highest correlation with a Hamilton Depression Rating Scale to determine the subject's facial depression level including: depressed or non-depressed, wherein psychiatrist diagnoses the Hamilton Depression Rating Scale.

Furthermore, a Courtauld Emotional Control Scale is provided in the depression detection system to assess the subject's emotional suppression level, and if the subject shows high emotional suppression, the depression level requires further assessment.

In addition, the depression detection further comprises an Event-Driven Depression Tendency Warning model version II and a Gaussian Process Regression model, wherein the Event-Driven Depression Tendency Warning model version II reads a text data transformed from the subject's video recording of personal stressful event description, and generates the subject's semantic depression level including: depressed or non-depressed after analyzing and assessing; after that, the Gaussian Process Regression model read the facial depression level and the semantic depression level in order to generate an integrated depression level including: depressed or non-depressed, wherein the weight of semantic depression level is higher than the weight of the facial depression level because the subject shows high emotional suppression.

### Experimental Example 1

The valence-arousal model in embodiment 1 is verified using 1999 facial images sourced from AffectNet database, and also another 287651 facial images are used for training the valence-arousal model that combines seven original valence-arousal coordinates of basic emotions including: Surprise, Happy, Neutral, Fear, Sad, Anger and Disgust into four standard coordinates of emotions including: happy, neutral, sad and anger, wherein the standard coordinate happy includes the original valence-arousal coordinate Surprise and Happy, and other standard coordinates are the same as the original valence-arousal coordinate with the same emotion; after training the valence-arousal model, a regression returns 73.8% of total root-mean-square error, 0.286 of arousal root-mean-square error and 0.247 of valence root-mean-square error.

### Experimental Example 2

To train and verify an analysis model which is the Random Forest model and Multilayer Perceptron, 74 subjects' video recordings, obtained using method of Embodiment 1, are input into the analysis model, generating an accuracy of 77%, a precision of 71.4%, an F-score of 74%, and a recall of 76.9%, and validity is established simultaneously.

### Experimental Example 3

To train and verify the Event-Driven Depression Tendency Warning model version II, subjects' semantic recordings obtained using method of Embodiment 1 are input thereto, wherein the depression score of the semantic record in the Event-Driven Depression Tendency Warning model version II is evaluated according to Brief Symptom Rating Scale, thereby generating an accuracy of 64.5%, a precision of 68.9%, an f-score of 73.8% and a recall of 79.5%.

### Experimental Example 4

Regarding high emotional suppression subjects, the precision of the integrated depression level shows 77% after combining the facial depression level and the semantic depression level, followed by weighting and analyzing as described in Embodiment 1.

### Experimental Example 5

A relevance analysis is performed between the 62 subjects' facial depression level and the semantic depression level obtained using the method in Embodiment 1 and the Hamilton Depression Rating Scale diagnosed by a psychiatrist. Referring to the following TABLE 1, when score of the Hamilton Depression Rating Scale is larger than 7, the facial depression level and the semantic depression level show moderate correlation with the Hamilton Depression Rating Scale, wherein the facial depression level exhibit 0.434 of r-value and p-value less than 0.05, the semantic depression level exhibit 0.370 of r-value and p-value less than 0.05, which indicates that both methods to obtain facial depression level and semantic depression level are stable.

TABLE 1

| | Total | HDRS ≥ 7 | HRDS < 7 |
|---|---|---|---|
| | (N = 62) | (n = 39) | (n = 23) |
| | Average (Standard Deviation) | | |
| Age | 42.29(15.52) | 36.85(15.02) | 51.52(11.68) |
| HDRS score | 7.71(4.71) | 10.74(2.87) | 2.57(1.78) |

Analysis Model numbers (%)

| | | | |
|---|---|---|---|
| Depressed | 42(67.7) | 30(76.9) | 12(52.2) |
| Non-depressed | 20(32.3) | 9(23.1) | 11(47.8) |

Semantic Analysis Model

| | | | |
|---|---|---|---|
| Depressed | 45(72.6) | 31(79.5) | 14(47.8) |
| Non-depressed | 17(27.4) | 8(20.5) | 9(39.1) |

### Experimental Example 6

Following the assessment of the correlation between 44 patients' facial depression level and semantic depression level obtained using the method in embodiment 1 and different depression measurement tools, including a Hamilton Depression Rating Scale and a Beck Depression Inventory, referring to TABLE 2, it is observed that the facial depression level has significant relevance with the Hamilton Depression Rating Scale diagnosed by a psychiatrist. The integrated depression level, using a more meticulous analytic procedure, has medium to high relevance with both the Hamilton Depression Rating Scale and the Beck Depression Inventory.

In addition, depression rate of the integrated depression level is higher than those of the depression measurement tools including HDRS and BDI-II, thereby showing higher sensitivity to subtle emotions; the analysis model in TABLE 1 and 2 corresponds to the Random Forest model and Multilayer perceptron, the semantic analysis model in TABLE 1 and 2 corresponds to the Event-Driven Depression Tendency Warning model version II, and the model combined facial and semantic analysis in TABLE 1 and 2 corresponds to the Gaussian Process Regression.

TABLE 2

| Depression Detection Outcome | HDRS | BDI-II |
|---|---|---|
| Analysis Model | 0.422** | 0.226 |
| Happy | 0.112 | 0.160 |
| Neutral | −0.126 | −0.200 |
| Anger | 0.265 | 0.176 |
| Sad | −0.076 | −0.174 |
| Semantic Analysis Model | 0.135 | 0.231 |
| Event | 0.010 | 0.107 |
| Mood | 0.085 | 0.133 |
| Symptom | 0.304* | 0.307* |
| Thought | 0.208 | 0.367* |
| Psychological Factors | 0.101 | 0.271 |
| Model combined Facial and | 0.394** | 0.368* |
| Semantic analysis | | |

Spearman's correlation:
*means: p-value lower than 0.05 and
**means: p-value lower than 0.01.

### Experimental Example 7

Regression analysis is performed between the integrated depression level obtained using the method in embodiment 1 and the Hamilton Depression Rating Scale diagnosed by a psychiatrist. Referring to TABLE 3, from a perspective of depression detection on subjects with cancer psychological adjustment, the integrated depression level has significant relevance with the Hamilton Depression Rating Scale, and there is a 20% R², which is called the coefficient of determination.

TABLE 3

| | B | R | R² | ΔR² | F | t |
|---|---|---|---|---|---|---|
| Model 1 | | 0.467 | 0.218 | 0.218 | 11.738** | |
| (content) | | | | | | 9.738*** |
| Model combined | −0.467 | | | | | −3.426** |
| Facial and | | | | | | |
| Semantic analysis | | | | | | |
| Model 2 | | 0.609 | 0.307 | 0.152 | 9.894 | |
| (content) | | | | | | 10.914*** |
| Model combined | −0.356 | | | | | −2.604* |
| Facial and | | | | | | |
| Semantic analysis | | | | | | |
| Psychological | −0.390 | | | | | −3.145* |
| Factors | | | | | | |

Spearman's correlation:
*means: p-value lower than 0.05,
**means: p-value lower than 0.01, and
***means: p-value lower than 0.001.
B: Regression Coefficient, R²: Coefficient of Determination, F and t: Test Statistic.

### Experimental Example 8

Referring to TABLE 4, it is observed that the semantic depression level obtained from the semantic analysis step remains accurate among the subjects with high emotional suppression, and there is a significant difference between the subjects with high emotional suppression and the subjects without high emotional suppression; therefore, analysis of the integrated depression step is able to promote accuracy of depression detection, and especially when increasing weight of the semantic depression level for the subjects with high emotional suppression.

TABLE 4

| | | CECS | | | Anger in CECS | | | Depression in CECS | | | Anxiety in CECS | | |
|---|---|---|---|---|---|---|---|---|---|---|---|---|---|
| | | G1 | G2 | | G1 | G2 | | G1 | G2 | | G1 | G2 | |
| | | Total(N = 44) | | | | | | | | | | | |
| | | (n = 20) | (n = 24) | p | (n = 21) | (n = 23) | p | (n = 22) | (n = 22) | p | (n = 20) | (n = 24) | p |
| | | (Range)M ± SD | | | | | | | | | | | |
| | | M ± SD | | | M ± SD | | | M ± SD | | | M ± SD | | |
| Analysis | (0-1) | 0.35 ± | 0.54 ± | 0.213 | 0.38 ± | 0.52 ± | 0.360 | 0.36 ± | 0.55 ± | 0.236 | 0.38 ± | 0.52 ± | 0.360 |
| Model | 0.45 ± | 0.49 | 0.51 | | 0.49 | 0.51 | | 0.49 | 0.51 | | 0.49 | 0.51 | |
| | 0.50 | | | | | | | | | | | | |
| Happy | (0-0.45) | 0.11 ± | 0.11 ± | 0.465 | 0.11 ± | 0.11 ± | 0.896 | 0.11 ± | 0.11 ± | 0.880 | 0.09 ± | 0.13 ± | 0.357 |
| | 0.11 ± | 0.09 | 0.13 | | 0.09 | 0.12 | | 0.11 | 0.11 | | 0.09 | 0.13 | |
| | 0.11 | | | | | | | | | | | | |
| Neutral | (0-0.93) | 0.49 ± | 0.43 ± | 0.502 | 0.44 ± | 0.48 ± | 0.624 | 0.49 ± | 0.43 ± | 0.483 | 0.45 ± | 0.46 ± | 0.931 |
| | 0.46 ± | 0.27 | 0.31 | | 0.29 | 0.30 | | 0.27 | 0.31 | | 0.31 | 0.28 | |
| | 0.29 | | | | | | | | | | | | |
| Anger | (0-0.99) | 0.14 ± | 0.18 ± | 0.358 | 0.16 ± | 0.17 ± | 0.835 | 0.15 ± | 0.18 ± | 0.682 | 0.20 ± | 0.13 ± | 0.286 |
| | 0.16 ± | 0.22 | 0.24 | | 0.21 | 0.25 | | 0.25 | 0.20 | | 0.28 | 0.17 | |
| | 0.23 | | | | | | | | | | | | |
| Sad | (0-0.94) | 0.25 ± | 0.28 ± | 0.850 | 0.29 ± | 0.24 ± | 0.485 | 0.25 ± | 0.28 ± | 0.671 | 0.25 ± | 0.28 ± | 0.648 |
| | 0.16 ± | 0.21 | 0.28 | | 0.25 | 0.25 | | 0.22 | 0.27 | | 0.26 | 0.23 | |
| | 0.23 | | | | | | | | | | | | |
| Semantic | (0-1) | 0.45 ± | 0.75 ± | 0.046 | 0.48 ± | 0.74 ± | 0.079 | 0.45 ± | 0.77 ± | 0.031 | 0.57 ± | 0.65 ± | 0.593 |
| Analysis | 0.61 ± | 0.51 | 0.44 | | 0.21 | 0.45 | | 0.51 | 0.43 | | 0.51 | 0.49 | |
| Model | 0.49 | | | | | | | | | | | | |
| Event | (0-0.04) | 0.00 ± | 0.01 ± | 0.031 | 0.00 ± | 0.01 ± | 0.002 | 0.00 ± | 0.01 ± | 0.276 | 0.00 ± | 0.01 ± | 0.698 |
| | 0.00 ± | 0.00 | 0.01 | | 0.00 | 0.01 | | 0.01 | 0.01 | | 0.01 | 0.01 | |
| | 0.01 | | | | | | | | | | | | |
| Mood | (0-0.97) | 0.13 ± | 0.20 ± | 0.251 | 0.12 ± | 0.21 ± | 0.147 | 0.18 ± | 0.15 ± | 0.623 | 0.18 ± | 0.16 ± | 0.762 |
| | 0.17 ± | 0.23 | 0.19 | | 0.21 | 0.21 | | 0.26 | 0.14 | | 0.25 | 0.17 | |
| | 0.21 | | | | | | | | | | | | |
| Symptom | (0-5.01) | 0.12 ± | 0.54 ± | 0.072 | 0.33 ± | 0.36 ± | 0.914 | 0.33 ± | 0.36 ± | 0.902 | 0.40 ± | 0.29 ± | 0.663 |
| | 0.35 ± | 0.15 | 1.10 | | 0.57 | 10.03 | | 1.05 | 0.56 | | 1.07 | 0.56 | |
| | 0.84 | | | | | | | | | | | | |
| Thought | (0-0.78) | 0.00 ± | 0.05 ± | 0.198 | 0.04 ± | 0.01 ± | 0.542 | 0.00 ± | 0.05 ± | 0.173 | 0.00 ± | 0.05 ± | 0.195 |
| | 0.03 ± | 0.01 | 0.16 | | 0.17 | 0.05 | | 0.00 | 0.17 | | 0.01 | 0.17 | |
| | 0.12 | | | | | | | | | | | | |
| Psychological | (0-0.08) | 0.01 ± | 0.02 ± | 0.138 | 0.01 ± | 0.01 ± | 0.956 | 0.01 ± | 0.02 ± | 0.088 | 0.01 ± | 0.02 ± | 0.093 |
| Factors | 0.01 ± | 0.01 | 0.02 | | 0.02 | 0.01 | | 0.01 | 0.02 | | 0.00 | 0.02 | |
| | 0.02 | | | | | | | | | | | | |

CECS: Courtauld Emotional Control Scale; G1: lower suppression; G2: higher suppression; M: average; SD: Standard Deviation; Spearman's correlation:
*means: p-value lower than 0.05 and
**means: p-value lower than 0.01.

The method of depression detection and system thereof in the present disclosure provides more accurate and personalized depression detection about emotional expression, emotional suppression, and physical and mental health for women with breast cancer during or after chemotherapy, thereby enabling precautionary and preventative intervention in treatment.

Besides, the accuracy of depression detection is increased when performing analysis combining facial depression and semantic depression that can effectively distinguish explicit emotional features of subjects with high emotional suppression.

On the other hand, in addition to psychological adjustment for subjects with cancer, the method in the present disclosure can also identify emotional features revealing psychological adjustment disorder and detect early depression in subjects with other high-risk diseases, such as high blood pressure, diabetes, and nephropathy, in order to maintain their mental health.

What is claimed is:

1. A method of depression detection, comprising:
a face marking step: marking a facial image to obtain a plurality of facial features, wherein the facial image is an image sequence obtained by processing a subject's video recording frame by frame;
an emotion classification and calculation step: inputting the plurality of facial features into an emotional classification model to generate a plurality of corresponding depression coordinates, and calculating the plurality of corresponding depression coordinates in accordance with a standard coordinate to generate a plurality of features;
a depression identification step: performing a statistical analysis on the plurality of features to generate a statistical feature, extracting the statistical feature having the highest correlation with a depression measurement tool as a highest correlated feature, and inputting the highest correlated feature into an analysis model to generate a facial depression level comprising: depressed and non-depressed; and
a verification step: when the facial depression level is the non-depressed, determining an emotional suppression level of the subject by a suppression measurement tool, wherein the facial depression level is true when the emotional suppression level shows no high emotional suppression.

2. The method of depression detection according to claim 1, wherein the video recording further comprises a semantic record, and the method of depression detection further comprises steps of:
a semantic analysis step: inputting the semantic record into a semantic analysis model to generate a semantic depression level; and
an integrated depression step: inputting the facial depression level and the semantic depression level into an integrated model to generate an integrated depression level, wherein when the facial depression level is the non-depressed and the emotional suppression level shows high emotional suppression, a weight of the semantic depression level is larger than a weight of the facial depression level.

3. The method of depression detection according to claim 1, wherein the video recording is a frontal video of the subject describing self-mentation for a duration, and the duration is 5 to 30 minutes.

4. The method of depression detection according to claim 1, wherein the emotional classification model comprises: a Valence-Arousal model, a Valence-Arousal model with POSTER++, a Multimodal model, a Convolutional Neural Network, a Long Short-Term Memory model, or any combination of two or more thereof.

5. The method of depression detection according to claim 1, wherein the standard coordinate comprises: a first coordinate, a second coordinate, a third coordinate and a fourth coordinate, wherein the first coordinate combines and relocates a coordinate of basic emotion Happy with a coordinate of basic emotion Surprise; the second coordinate, the third coordinate and the fourth coordinate correspond to a coordinate of basic emotion Neutral, a coordinate of basic emotion Sad and a coordinate of basic emotion Anger, respectively.

6. The method of depression detection according to claim 1, wherein the depression measurement tool comprises: a Hamilton Depression Rating Scale, a Beck Depression Inventory, a Patient Health Questionnaire, or a Taiwanese Depression Scale.

7. The method of depression detection according to claim 1, wherein the suppression measurement tool comprises: a Courtauld Emotional Control Scale, an Emotional Regulation Questionnaire, or an Emotional Expressivity Scale.

8. The method of depression detection according to claim 1, wherein the analysis model comprises: an Ensemble Voting Classifier, a Random Forest model, a Multilayer Perceptron, a Decision Tree, a Support Vector Machine, an Artificial Neural Network, a Convolutional Neural Network, or any combination of two or more thereof.

9. The method of depression detection according to claim 2, wherein the semantic analysis model comprises: an Event-Driven Depression Tendency Warning model, an Event-Driven Depression Tendency Warning model version II or a Python senti_c package; the integrated model comprises: a Gaussian Process Regression model or a Bayesian Neural Network.

10. The method of depression detection according to claim 1, wherein the method for obtaining the plurality of facial features comprises: a Convolutional Neural Network, an Open Computer Vision Library, a Py-Feat, an OpenFace, an Active Appearance Model, or any combination of two or more thereof.

11. A depression detection system, comprising:
an emotional classification model configured to:
read a plurality of facial features to generate a plurality of corresponding depression coordinates, and calculate the plurality of corresponding depression coordinates in accordance with a standard coordinate to generate a plurality of features, wherein the plurality of facial features is obtained by marking an image sequence of a subject's face, which is a video recording processed frame by frame; and
an analysis model configured to:
read a highest correlated feature to determine a facial depression level, wherein the highest correlated feature is a statistical feature exhibiting the highest correlation with a depression measurement tool, and the statistical feature is derived through a statistical analysis of the plurality of features, wherein the facial depression level comprises: depressed and non-depressed, and while the facial depression level is the non-depressed, a suppression measurement tool is provided to determine an emotional suppression level of the subject, wherein the facial depression level is true when the emotional suppression level shows no high emotional suppression.

12. The depression detection system according to claim 11, further comprises:
a semantic analysis model configured to:
read a semantic record and generate a semantic depression level, wherein the semantic record is obtained from processing the video recording; and
an integrated model configured to:
read the facial depression level and the semantic depression level to generate an integrated depression level, wherein when the facial depression level is the non-depressed and the emotional suppression level shows high emotional suppression, a weight of the semantic depression level is larger than a weight of the facial depression level.

13. The depression detection system according to claim 11, wherein the video recording is a frontal video of the subject describing self-mentation for a duration, and the duration is 5 to 30 minutes.

14. The depression detection system according to claim 11, wherein the emotional classification model comprises: a Valence-Arousal model, a Valence-Arousal model with POSTER++, a Multimodal model, a Convolutional Neural Network, a Long Short-Term Memory model, or any combination of two or more thereof.

15. The depression detection system according to claim 11, wherein the standard coordinate comprises: a first coordinate, a second coordinate, a third coordinate and a fourth coordinate, wherein the first coordinate combines and relocates a coordinate of basic emotion Happy with a coordinate of basic emotion Surprise; the second coordinate, the third coordinate and the fourth coordinate correspond to a coordinate of basic emotion Neutral, a coordinate of basic emotion Sad and a coordinate of basic emotion Anger, respectively.

16. The depression detection system according to claim 11, wherein the depression measurement tool comprises: a Hamilton Depression Rating Scale, a Beck Depression Inventory, a Patient Health Questionnaire, or a Taiwanese Depression Scale.

17. The depression detection system according to claim 11, wherein the suppression measurement tool comprises: a Courtauld Emotional Control Scale, an Emotional Regulation Questionnaire, or an Emotional Expressivity Scale.

18. The depression detection system according to claim 11, wherein the analysis model comprises: an Ensemble Voting Classifier, a Random Forest model, a Multilayer Perceptron, a Decision Tree, a Support Vector Machine, an Artificial Neural Network, a Convolutional Neural Network, or any combination of two or more thereof.

19. The depression detection system according to claim 12, wherein the semantic analysis model comprises: an Event-Driven Depression Tendency Warning model, an Event-Driven Depression Tendency Warning model version II or a Python senti_c package; the integrated model comprises: a Gaussian Process Regression model or a Bayesian Neural Network.

20. The depression detection system according to claim 11, wherein the method of obtaining the plurality of facial features comprises: a Convolutional Neural Network, an Open Computer Vision Library, a Py-Feat, an OpenFace, an Active Appearance Model, or any combination of two or more thereof.
